(19) United States
(12) Patent Application Publication
(10) Pub. No.: US 2008/0208120 A1
(43) Pub. Date: Aug. 28, 2008

(54) INSTRUMENT FOR INJECTIONS WITH RETRACTABLE NEEDLE FOLLOWING USE

(76) Inventor: Fabio BOLDINI, Puegnago Sul Garda (Brescia) (IT)

Correspondence Address: MCGLEW & TUTTLE, PC, P.O. BOX 9227, SCARBOROUGH STATION, SCARBOROUGH, NY, 10510-9227, US

(21) Appl. No.: 12/035,730

(22) Filed: Feb. 22, 2008

(30) Foreign Application Priority Data
Feb. 26, 2007 (IT) BS2007A000024

(51) Int. Cl. A61M 5/50

(52) U.S. Cl. 604/110

(57) ABSTRACT

The invention concerns an instrument for injections that comprises a needle (12) fixed to a needle-holder block (19) positioned in a connecting element (15) that is attached to the neck of a container of a medicinal substance or to the cylinder of a syringe. The block with needle moves between a forward position in which the needle extends forward beyond the connecting element and a retracted position in which the needle is completely inside the connecting element, where the forward position of the needle is established by a catch (21) moving between and interception position and a release position of said needle-holder block (19) while the retracted position of the needle is provoked by a return spring (20) in answer to the release of the needle-holder block on the part of the catch. FIG. 7

### FIELD OF THE INVENTION

This invention concerns the medical field and refers in particular to an instrument for intramuscular and subcutaneous injections of the type comprised of a ampoule or phial of medicinal substances connected to a needle for injections.

### STATE OF THE ART

In the field taken into consideration, an instrument for injections that comprises a container shaped as an ampoule or phial, of a flexible material, preloaded or loadable, and a needle for injections supported by a connecting element is already known. The medicinal container has a neck and the needle is coupled to said neck either by being screwed or pressed into it.

Initially, the neck of the container is sealed, but it may be perforated; the needle has a rear perforating end facing towards the neck; the connecting element is constrained in a first position relatively to the neck of the container so that the rear perforating end of the needle is at a distance from the neck so as not to perforate it; the needle is enclosed in a protective cap. To inject the medicinal substance, the connecting element is tightly engaged on the neck of the container in a second position so that the perforating end of the needle will perforate the neck and thus be in communication with the inside of the container. Then, once the protective cap has been removed, the injection can be executed by compressing the container obliging the medicinal substance to exit from the needle towards the patient.

However, after the injection and when it has been extracted from the patient, the needle remains in a fixed position and exposed with the risk of injuring/contaminating anybody accidentally coming into contact with it, even only when replacing the protective cap.

Technically and commercially, syringes with a retractable needle following use so as to avoid the risk of contamination are well known, but as far as we know the retractable technique of the needle has never been applied to instruments of the type described above.

### OBJECT AND SUMMARY OF THE INVENTION

The object of this invention is to remedy this shortcoming and, therefore, create the conditions to protect against any contact and risk of contamination those instruments for injections that use a compressible, preloaded or loadable container with a medicinal substance, connected to a needle for injections moving between a first neutral position and a second perforating position of the container.

Such an object is reached, according to the invention, with a container of medicinal substance according to the preface of claim 1, wherein the needle is fixed using a needle-holder block provided in the connection element and moving between a forward position in which the needle extends forward beyond the connecting element and a retracted position in which the needle is fully retracted inside the connecting element, and wherein the forward position of the needle-holder block is set by a catch moving between an engaging and a release position of said needle-holder block with said block and the retracted position is originated by a return spring in response to the release of said needle-holder block by the catch.

The container can be produced both complete with medicinal substance, or empty, to be loaded later by the end operator: by compressing the body of the container to make the air it contains exit and by taking advantage of the consequent return to fill it with medicinal substance.

### BRIEF DESCRIPTION OF THE DRAWINGS

The invention will however be illustrated in greater detail in the description to follow in reference to the attached indicative and not limiting drawings, in which;

FIG. 1 shows a view in perspective of the instrument for injections with needle protected by a cap;

FIGS. 2 and 3 show two exterior views of the instrument in FIG. 1 in right-angled directions;

FIGS. 4 and 5 show two similar external views of the instrument with internal parts sketched in dotted lines to indicate the arrangement:;

FIG. 6 shows a longitudinal section according to arrows A-A in FIG. 2; and

FIG. 7 shows a section plane of the instrument with needle retracted.

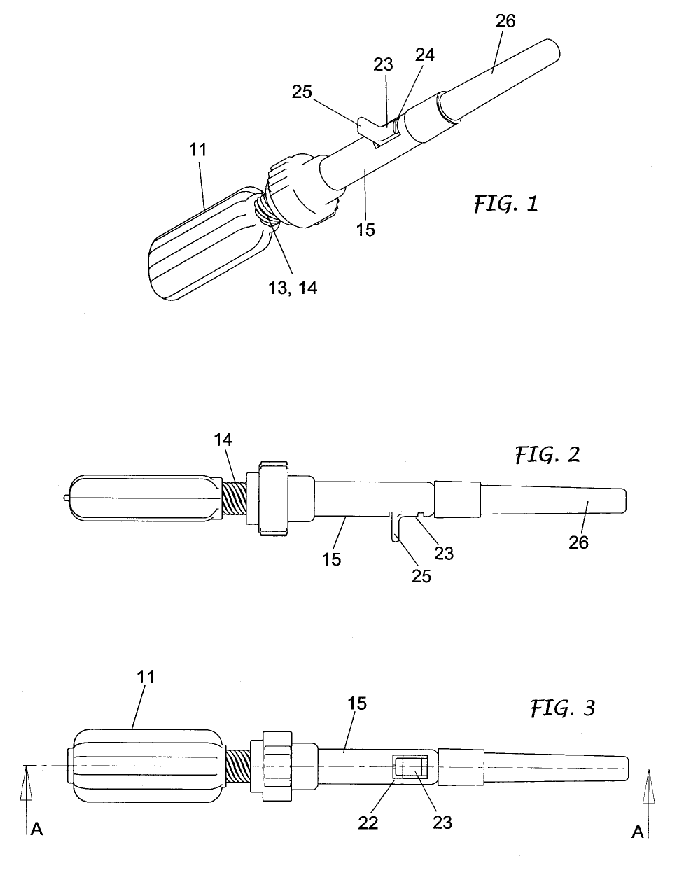

### DETAILED DESCRIPTION OF THE INVENTION

As represented, the instrument for injections comprises a container 11 made of a flexible material in the shape of a compressible ampoule or phial and a needle for injections 12 associated with it. The container 11 is filled with an injectable medicinal substance and has a neck 13 that is sealed and has a threaded external section 14. The needle 12 is attached to the neck 13 of the container by means of a connecting device 15 with a perforated distal part 16 and a proximal part 17 that is threaded and connects by screwing into the threaded section 14 of said neck. The needle 12 has a front end 12′ facing towards the distal end 16 of the connecting element and a rear end 12″ facing towards the neck 13 of the container 11.

More precisely, the connecting element 15 internally forms an axial housing 18, and the needle 12 is fixed to a needle-holder block 19, that is housed inside with the possibility of sliding in the housing 18 of said connecting element 15. The needle-holder block 19, including the needle 12, are able to move in said housing 18 between a forward position towards the distal end 16 and a retracted position towards the proximal part 17 of the connecting element 15.

In the example illustrated, after, between the needle-holder block 19 and the distal end 16 of the connecting element 15 there is a compressible return spring 20 provided.

Worthy of note in this case is that the needle-holder block 19 and the return spring can be both simply associated individual components, and elements preventively constrained to each other, for example by co-moulding of the needle-holder block 19, needle 12 and spring 20.

In any case, when the needle-holder block 19 is in the forward position the needle 12 protrudes from the perforated distal end 16 of the connecting element 15. In this position, the return spring 20 is compressed between the needle-holder block 19 and the distal part 16 of the connecting element, and the needle-holder block 19 is held in position by means of a catch 21, positioned at level with the opening 22 in one side of the connecting element 15 and movable between an active blocking position and an inactive release position of the needle-holder block.

The catch 21 is part of a lever 23 that can be integrally machined with the connecting element 15 and connected with the latter by means of an oscillation fulcrum 24. Furthermore, the catch 21 can be devised so that when it is in the active blocking position it engages with the rear of the needle-holder block 19 as shown in the drawings, or alternatively with an intermediate part of said recess, not shown.

The lever 23 normally remains in a state in which the catch 21 is in the active blocking position of the needle-holder block 19, but can be manually moved out of said position by means of a grip 25 it is an integral part of, so as to move the catch 21 away from the needle-holder block 19 and thus release the latter so it can move backward towards the proximal end 17 of the connecting element 15.

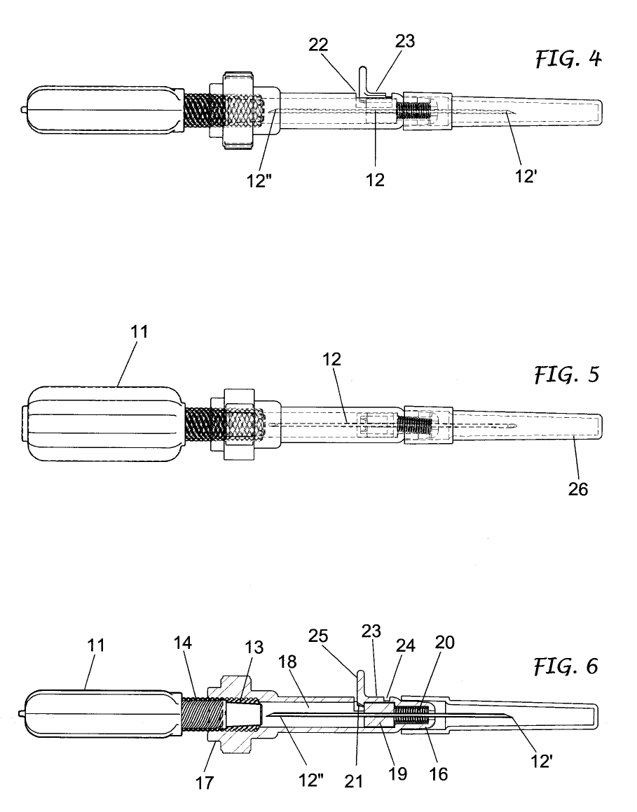

Initially, the container 11 is filled with a medicinal substance and its neck 13 is sealed. The needle-holder block 19 is held in its forward position with the needle protruding from the distal part 16 of the connecting element 15. This connecting element 15 is screwed to the container, but only partially until the rear end 12″ of the needle reaches the neck 13. The portion of the needle 12 that protrudes from the connecting element 15 is subsequently enclosed in a protective cap 26—FIGS. 4-6.

When administering the medicinal substance to a patient the connecting element 15 is screwed tightly onto the neck 13 of the container 11 so that the rear end 12″ of the needle 12 will perforate the neck. In this way the needle is now in communication with the inside of the container 11 and after removing the protective cap 26, front end 12′ of the needle can be inserted into the body of the patient to inject the medicinal substance.

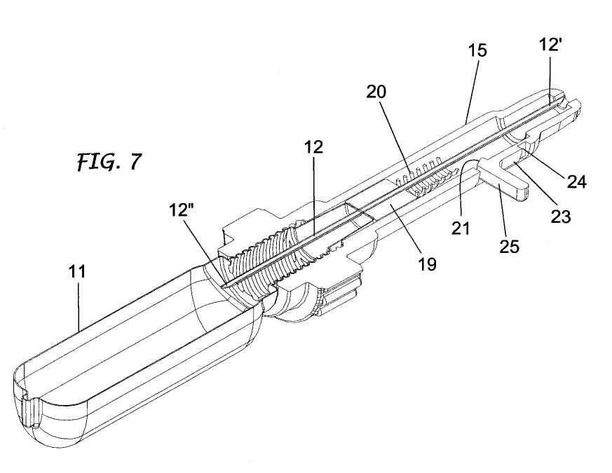

Once the injection has been carried out and the needle extracted from the body of the patient, the operator can use the lever 23 to move the catch 21 away from the needle-holder block 19 and release the latter. Consequently, driven by the return spring 20, the needle-holder block 19 moves back towards the neck 13 of the container until the needle is completely inside the connecting element—FIG. 7—and therefore in a protected condition without the risk of contact with its front end.

It should also be understood that small changes to details can be added to the group described above without however moving outside the sphere of the invention and that the system whereby the needle moves back inside the connecting element may be suitable and applied also to normal syringes and to every other device that comprises a needle for infusions or injections where it would be ideal if it could move back and be concealed after use.

1. An instrument for injections with a retractable needle after use, the instrument comprising:
a container for a medicinal substance in the shape of an ampoule or vial made of a flexible material, and a needle for injections supported by a connecting element, said container having a sealed neck with external threading, said needle being connected to said neck by screwing said connecting element onto said neck, said needle having a front end for protruding forward from the connecting element and a rear end facing towards the neck of said container, wherein initially the connecting element is only partially screwed to the neck so that the rear end of the needle is distant from said neck, said connecting element being tightly screwed to the neck of the container so that the rear end of the needle perforates the neck when the instrument is used for an injection, said needle being fixed to a needle-holder block provided in said connecting element and moving between a forward position in which the needle extends forward beyond the connecting element and a back position in which the needle is completely withdrawn inside the connecting element, wherein the forward position of the needle-holder block is established by a catch moving between an interception position and a release position of said the needle-holder block, and the retracted position of said needle-holder block is provoked by the return spring in response to the release of the needle-holder block on the part of the catch.

2. Instrument for injections according to claim 1, wherein said connecting element has a perforated distal end and a threaded proximal end that connects to the neck of the container, in which the needle-holder block is positioned and slides in said housing between said distal and proximal end, and in which the return spring is placed and loaded between the needle-holder block and the distal end of the connecting element when the block is held in the forward position.

3. Instrument for injections according to claim 1, wherein the catch of the needle-holder block is part of a lever connected to the connecting element by means of an oscillating fulcrum and provided with a gripper tang so as to shift the catch from the interception position to the release position of the needle-holder block.

4. Instrument for injections according to claim 3, wherein the catch is positioned level with an opening in one side of the connecting element engaging with the rear of the needle-holder block to hold the needle-holder block in the forward position.

5. Instrument for injections according to claim 3, wherein the catch is positioned level with an opening in one side of the connecting element engaging with an intermediate part of the needle-holder block.

6. Instrument for injections according to claim 1, wherein the needle-holder block and the return spring are individually associated elements.

7. Instrument for injections according to claim 1, wherein the needle-holder block and the return spring are preassembled by co-molding of said block on the end of said spring.

8. Safety system to prevent the re-use of a needle for injections so as to avoid injury and/or infection of anybody who comes into contact with the needle after the needle has been used, applicable to ampoules or syringes and including a connecting element complete with needle, needle-holder block, return spring, blocking/releasing needle system and protective cap according to any of the previous claims.

9. Instrument for injections according to claim 2, wherein the catch of the needle-holder block is part of a lever connected to the connecting element by means of an oscillating fulcrum and provided with a gripper tang so as to shift the catch from the interception position to the release position of the needle-holder block.
